SEQUENCE LISTING (8 sequences)

SEQ ID NO: 1 - protein, 192 aa, SARS-CoV-2 virus
PNITNLCPFGEVFNATRFASVYAWNRKRISNCVADYSVLYNSASFSTFKCYGVSPTKLNDLCFTNVYADSFVIRGDEVRQIAPGQTGKIADYNYKLPDDFTGCVIAWNSNNLDSKVGGNYNYLYRLFRKSNLKPFERDISTEIYQAGSTPCNGVEGFNCYFPLQSYGFQPTNGVGYQPYRVVVLSFELLHAP

SEQ ID NO: 2 - protein, 192 aa, SARS-CoV-2 virus
PNITNLSPFGEVFNATRFASVYAWNRKRISNSVADYSVLYNSASFSTFKSYGVSPTKLNDLSFTNVYADSFVIRGDEVRQIAPGQTGKIADYNYKLPDDFTGSVIAWNSNNLDSKVGGNYNYLYRLFRKSNLKPFERDISTEIYQAGSTPSNGVEGFNSYFPLQSYGFQPTNGVGYQPYRVVVLSFELLHAP

SEQ ID NO: 3 - DNA, 576 bp, Artificial Sequence
  Genetic coding for SEQ ID No 1
ccgaacattaccaacctgtgcccgtttggcgaagtgtttaacgcgacccgctttgcgagcgtgtatgcgtggaaccgcaaacgcattagcaactgcgtggcggattatagcgtgctgtataacagcgcgagctttagcacctttaaatgctatggcgtgagcccgaccaaactgaacgatctgtgctttaccaacgtgtatgcggatagctttgtgattcgcggcgatgaagtgcgccagattgcgccgggccagaccggcaaaattgcggattataactataaactgccggatgattttaccggctgcgtgattgcgtggaacagcaacaacctggatagcaaagtgggcggcaactataactatctgtatcgcctgtttcgcaaaagcaacctgaaaccgtttgaacgcgatattagcaccgaaatttatcaggcgggcagcaccccgtgcaacggcgtggaaggctttaactgctattttccgctgcagagctatggctttcagccgaccaacggcgtgggctatcagccgtatcgcgtggtggtgctgagctttgaactgctgcatgcgccg

SEQ ID NO: 4 - DNA, 576 bp, Artificial Sequence
  Genetic coding for SEQ ID No 2
ccgaacatcaccaacctgtctccgttcggtgaagttttcaacgcgacccgtttcgcgtctgtttacgcgtggaaccgtaaacgtatctctaactctgttgcggactactctgttctgtacaactctgcgtctttctctaccttcaaatcttacggtgtttctccgaccaaactgaacgacctgtctttcaccaacgtttacgcggactctttcgttatccgtggtgacgaagttcgtcagatcgcgccgggtcagaccggtaaaatcgcggactacaactacaaactgccggacgacttcaccggttctgttatcgcgtggaactctaacaacctggactctaaagttggtggtaactacaactacctgtaccgtctgttccgtaaatctaacctgaaaccgttcgaacgtgacatctctaccgaaatctaccaggcgggttctaccccgtctaacggtgttgaaggtttcaactcttacttcccgctgcagtcttacggtttccagccgaccaacggtgttggttaccagccgtaccgtgttgttgttctgtctttcgaactgctgcacgcgccg

SEQ ID NO: 5 - protein, 1273 aa, SARS-CoV-2 virus
MFVFLVLLPLVSSQCVNLTTRTQLPPAYTNSFTRGVYYPDKVFRSSVLHSTQDLFLPFFSNVTWFHAIHVSGTNGTKRFDNPVLPFNDGVYFASTEKSNIIRGWIFGTTLDSKTQSLLIVNNATNVVIKVCEFQFCNDPFLGVYYHKNNKSWMESEFRVYSSANNCTFEYVSQPFLMDLEGKQGNFKNLREFVFKNIDGYFKIYSKHTPINLVRDLPQGFSALEPLVDLPIGINITRFQTLLALHRSYLTPGDSSSGWTAGAAAYYVGYLQPRTFLLKYNENGTITDAVDCALDPLSETKCTLKSFTVEKGIYQTSNFRVQPTESIVRFPNITNLCPFGEVFNATRFASVYAWNRKRISNCVADYSVLYNSASFSTFKCYGVSPTKLNDLCFTNVYADSFVIRGDEVRQIAPGQTGKIADYNYKLPDDFTGCVIAWNSNNLDSKVGGNYNYLYRLFRKSNLKPFERDISTEIYQAGSTPCNGVEGFNCYFPLQSYGFQPTNGVGYQPYRVVVLSFELLHAPATVCGPKKSTNLVKNKCVNFNFNGLTGTGVLTESNKKFLPFQQFGRDIADTTDAVRDPQTLEILDITPCSFGGVSVITPGTNTSNQVAVLYQDVNCTEVPVAIHADQLTPTWRVYSTGSNVFQTRAGCLIGAEHVNNSYECDIPIGAGICASYQTQTNSPRRARSVASQSIIAYTMSLGAENSVAYSNNSIAIPTNFTISVTTEILPVSMTKTSVDCTMYICGDSTECSNLLLQYGSFCTQLNRALTGIAVEQDKNTQEVFAQVKQIYKTPPIKDFGGFNFSQILPDPSKPSKRSFIEDLLFNKVTLADAGFIKQYGDCLGDIAARDLICAQKFNGLTVLPPLLTDEMIAQYTSALLAGTITSGWTFGAGAALQIPFAMQMAYRFNGIGVTQNVLYENQKLIANQFNSAIGKIQDSLSSTASALGKLQDVVNQNAQALNTLVKQLSSNFGAISSVLNDILSRLDKVEAEVQIDRLITGRLQSLQTYVTQQLIRAAEIRASANLAATKMSECVLGQSKRVDFCGKGYHLMSFPQSAPHGVVFLHVTYVPAQEKNFTTAPAICHDGKAHFPREGVFVSNGTHWFVTQRNFYEPQIITTDNTFVSGNCDVVIGIVNNTVYDPLQPELDSFKEELDKYFKNHTSPDVDLGDISGINASVVNIQKEIDRLNEVAKNLNESLIDLQELGKYEQYIKWPWYIWLGFIAGLIAIVMVTIMLCCMTSCCSCLKGCCSCGSCCKFDEDDSEPVLKGVKLHYT

SEQ ID NO: 6 - protein, 1273 aa, SARS-CoV-2 virus
MFVFLVLLPLVSSQCVNLTTRTQLPPAYTNSFTRGVYYPDKVFRSSVLHSTQDLFLPFFSNVTWFHAIHVSGTKGTKRFDNPVLPFNDGVYFASTEKSNIIRGWIFGTTLDSKTQSLLIVNNATNVVIKVCEFQFCNDPFLGVYYHKNNKSWMESEFRVYSSANNCTFEYVSQPFLMDLEGKQGNFKNLREFVFKNIDGYFKIYSKHTPINLVRDLPQGFSALEPLVDLPIGINITRFQTLLALHRSYLTPGDSSSGWTAGAAAYYVGYLQPRTFLLKYNENGTITDAVDCALDPLSETKCTLKSFTVEKGIYQTSNFRVQPTESIVRFPNITNLCPFGEVFNATRFASVYAWNRKRISNCVADYSVLYNSASFSTFKCYGVSPTKLNDLCFTNVYADSFVIRGDEVRQIAPGQTGKIADYNYKLPDDFTGCVIAWNSNNLDSKVGGNYNYLYRLFRKSNLKPFERDISTEIYQAGSTPCNGVEGFNCYFPLQSYGFQPTNGVGYQPYRVVVLSFELLHAPATVCGPKKSTNLVKNKCVNFNFNGLTGTGVLTESNKKFLPFQQFGRDIADTTDAVRDPQTLEILDITPCSFGGVSVITPGTNTSNQVAVLYQDVNCTEVPVAIHADQLTPTWRVYSTGSNVFQTRAGCLIGAEHVNNSYECDIPIGAGICASYQTQTNSPRRARSVASQSIIAYTMSLGAENSVAYSNNSIAIPTNFTISVTTEILPVSMTKTSVDCTMYICGDSTECSNLLLQYGSFCTQLNRALTGIAVEQDKNTQEVFAQVKQIYKTPPIKDFGGFNFSQILPDPSKPSKRSFIEDLLFNKVTLADAGFIKQYGDCLGDIAARDLICAQKFNGLTVLPPLLTDEMIAQYTSALLAGTITSGWTFGAGAALQIPFAMQMAYRFNGIGVTQNVLYENQKLIANQFNSAIGKIQDSLSSTASALGKLQDVVNQNAQALNTLVKQLSSNFGAISSVLNDILSRLDKVEAEVQIDRLITGRLQSLQTYVTQQLIRAAEIRASANLAATKMSECVLGQSKRVDFCGKGYHLMSFPQSAPHGVVFLHVTYVPAQEKNFTTAPAICHDGKAHFPREGVFVSNGTHWFVTQRNFYEPQIITTDNTFVSGNCDVVIGIVNNTVYDPLQPELDSFKEELDKYFKNHTSPDVDLGDISGINASVVNIQKEIDRLNEVAKNLNESLIDLQELGKYEQYIKWPWYIWLGFIAGLIAIVMVTIMLCCMTSCCSCLKGCCSCGSCCKFDEDDSEPVLKGVKLHYT

SEQ ID NO: 7 - protein, 291 aa, SARS-CoV-2 virus
TQNVLYENQKLIANQFNSAIGKIQDSLSSTASALGKLQDVVNQNAQALNTLVKQLSSNFGAISSVLNDILSRLDKVEAEVQIDRLITGRLQSLQTYVTQQLIRAAEIRASANLAATKMSECVLGQSKRVDFCGKGYHLMSFPQSAPHGVVFLHVTYVPAQEKNFTTAPAICHDGKAHFPREGVFVSNGTHWFVTQRNFYEPQIITTDNTFVSGNCDVVIGIVNNTVYDPLQPELDSFKEELDKYFKNHTSPDVDLGDISGINASVVNIQKEIDRLNEVAKNLNESLIDLQE

SEQ ID NO: 8 - protein, 291 aa, SARS-CoV-2 virus
TQNVLYENQKLIANQFNSAIGKIQDSLSSTASALGKLQDVVNQNAQALNTLVKQLSSNFGAISSVLNDILSRLDKVEAEVQIDRLITGRLQSLQTYVTQQLIRAAEIRASANLAATKMSESVLGQSKRVDFSGKGYHLMSFPQSAPHGVVFLHVTYVPAQEKNFTTAPAICHDGKAHFPREGVFVSNGTHWFVTQRNFYEPQIITTDNTFVSGNSDVVIGIVNNTVYDPLQPELDSFKEELDKYFKNHTSPDVDLGDISGINASVVNIQKEIDRLNEVAKNLNESLIDLQE

(19) United States
(12) Patent Application Publication
(10) Pub. No.: US 2023/0181711 A1
(43) Pub. Date: Jun. 15, 2023

(54) IMMUNOGENIC COMPOSITION COMPRISING AN ANTIGENIC MOIETY AND A LIPOSOMAL FORMULATION, METHOD OF PRODUCING THE COMPOSITION, THE COMPOSITION FOR USE AS A MEDICAMENT, IN PARTICULAR FOR USE AS A VACCINE

(71) Applicant: INNOMEDICA HOLDING AG, Zug (CH)

(72) Inventors: Stéfan Jonathan HALBHERR, Bern (CH); Camille PEITSCH, Bern (CH); Diego VON WERDT, Bern (CH)

(21) Appl. No.: 17/917,421

(22) PCT Filed: Apr. 7, 2021
(86) PCT No.: PCT/EP2021/059055

§ 371 Date: Oct. 6, 2022

(30) Foreign Application Priority Data
Apr. 7, 2020 (EP) 20168554.2

(51) Int. Cl. A61K 39/12

(52) U.S. Cl. CPC A61K 39/12; A61K 2039/55572

(57) ABSTRACT

The present invention concerns an immunogenic composition comprising (a) an antigenic moiety, preferably an antigenic moiety being or comprising an amino acid sequence corresponding to a surface protein domain of SARS-CoV-2 virus; and (b) a liposomal formulation as an adjuvant. More specifically, the antigenic moiety preferably is either the receptor binding domain RBD of Spike protein S of SARS-CoV-2 virus, or the HR domain of S₂ subunit of spike protein S of SARS-CoV-2 virus; or an immunogenic fragment thereof. The invention further relates to a method of producing an immunogenic composition and the use of such composition as a medicament.

The invention relates to the technical field of immunogenic compositions, in particular to the use of antigenic moieties in combination with liposomal formulations as an adjuvant. The invention further relates to a method for producing the composition, the composition for use as a medicament and in particular for use as a vaccine.

The concept of provoking a protective immune response in the body by means of vaccines in order to prepare for a future exposure to viruses has been a commonly used means in medicine for a long time. With the outbreak of a novel kind of the highly pathogenic corona viruses, the search for efficient vaccination has become even more urgent. Since the first outbreak of SARS-CoV in 2002, the medical and scientific community demonstrated excellent efforts in the structural analysis and understanding of mechanisms related to corona viruses. Despite these efforts, there are still no treatments or prophylactics available which would reliably mitigate the risks that corona viruses pose to human and animal health.

It is the purpose of the subject matter of this application to overcome the current shortcomings of the art. It is in particular a purpose to provide an immunogenic composition comprising an antigenic moiety and a liposomal formulation which induce an immune response against viruses, in particular viruses of the corona type. It is a further purpose of the present invention to suggest a method of producing the composition, to suggest the composition for use as a medicament and in particular for use as a vaccine.

The invention relates to an immunogenic composition comprising
(a) an antigenic moiety, preferably an antigenic moiety being or comprising an amino acid sequence corresponding to a surface protein domain of SARS-CoV-2 virus; and
(b) a liposomal formulation as an adjuvant.

The invention is based on the idea to use peptides which, at least in sections, are similar to the composition of surface proteins of a virus, such as SARS-CoV-2 virus, as an antigenic moiety in combination with liposomal formulations.

The invention in particular relates to an immunogenic composition as described above, wherein the antigenic moiety (a) is or comprises an amino acid sequence corresponding to
(i) the receptor binding domain RBD of Spike protein S of SARS-CoV-2 virus;
(ii) an immunogenic fragment of the said receptor binding domain RBD;
(iii) a sequence having at least 90%, preferably at least 95%, more preferably at least 98% sequence identity to the receptor binding domain RBD of Spike protein S of SARS-CoV-2 virus.

With regard to SARS-CoV-2 virus, of particular interest is the receptor binding domain RBD of the virus. The domain is essential for binding of the virus to the human angiotensin-converting enzyme (ACE2) receptor, thereby promoting host cell invasion. RBD has a strong binding affinity to a host cell receptor and hence creates a configuration for subsequent fusion of the viral and the cellular membranes.

In the case of corona virus SARS-CoV-2, the RBD forms part of structural spike protein S. In particular, sub-unit S₁ of the transmembrane spike S glycoprotein, that forms homotrimers protruding from the viral surface, is of interest (Walls et al, Structure, Function, and antigenicity of the SARS-CoV-2 Spike Glycoprotein, Cell (2020), https://doi.org/10.1016/ j.cell.2020.02. 058). The receptor binding domain RBD has been found to bind strongly to human and bat angiotensin-converting enzyme 2 (ACE2) receptors and therefore serves as a target for development of vaccines (W Tai et al., Characterization of the receptor-binding domain (RBD) of 2019 novel coronavirus: implication for the development of RBD protein as a viral attachment inhibitor and vaccine; Cell Mol Immunol (2020); https://doi.org/10.1038/s41423-020-0400-4). SARS-CoV-2 Spike S protein is surface exposed on virions an infected cell. S protein confers virion docking to cells and endocytosis as well as release of replication machinery into the cytoplasm. S is the most potent unit of immune attack. S has multiple domains of which the receptor binding domain (RBD) is most exposed. RBD is the most potent point of virus neutralization. Convalescent COVID-19 patients display high titers of RBD specific neutralizing antibodies, indicating the potency of the RBD antigen to induce protective humoral immunity. Moreover, it has recently been shown that anti-RBD antibodies do not promote antibody-dependent enhancement (ADE) of diseases, which highlights the safe use of the RBD antigen in subunit vaccines.

For the purposes of this invention, SARS-CoV-2 virus means the virus as described in detail by Gorbalenya et al. The species Severe acute respiratory syndrome-related coronavirus: classifying 2019-nCoV and naming it SARS-CoV-2. Nat. Microbiol. https://doi.org/10.1038/s41564-020-0695-z (2020). For the purposes of the invention, the receptor binding domain RBD of spike protein S of SARS-CoV-2 virus means the amino acid sequence as deposited in GenBank/SRA (https://www.ncbi.nlm.nih.gov/genbank/sars-cov-2-seqs/; retrieved 06.04.2020). The scope of the invention also extends to an immunogenic fragment thereof or a sequence having at least 90%, preferably at least 95%, more preferably at least 98% sequence identity to the said receptor binding domain RBD.

According to the invention, the immunogenic composition further comprises a liposomal formulation as an adjuvant. The authors have found that a combination of the antigenic moiety and the adjuvant induces an immune response that is not significantly different from the immune response induced by the antigenic moiety alone. The liposomal system, however, enhances the effect of the antigenic moiety by enhancing the immune response. The liposomal system therefore provides for better response throughout the population and/or reduces the dose to be administered / the side effects which might accompany the vaccination.

A further aspect of the invention relates to a composition as described above, wherein the antigenic moiety is or comprises an amino acid sequence selected from
(i) SEQ ID No 1;
(ii) an immunogenic fragment of the said sequence;
(iii) a sequence having at least 90%, preferably at least 95%, more preferably at least 98% sequence identity to said SEQ ID No 1.

By SEQ ID No 1 is meant the receptor binding domain RBD of the Italian variant of the Spike protein S of SARS-CoV-2 virus according to GenBank MT066156.1 (retrieved 04.04.2020). The full Spike protein S of SARS-CoV-2/INMI1/human/2020/ITA virus has the protein sequence listed under SEQ ID 5 (ITA).

The receptor binding domain (SEQ ID 1) forms part thereof. The receptor binding domain corresponds to amino acids 329 to 521 of the whole protein sequence as described in the reference database (https://www.ncbi.nlm.nih.gov/genbank/sars-cov-2-seqs/) .

By SEQ ID No 1 is also meant the receptor binding domain RBD of the Chinese variant of the Spike protein S of SARS-CoV-2 virus according to GenBank MT226610.1 (retrieved 06.04.2020). The Spike protein S of SARS-CoV-2/KMS1/human/2020/CHN virus has the protein sequence as provided under SEQ ID 6 (CHN).

The receptor binding domain (SEQ ID 1) forms part thereof. The receptor binding domain corresponds to amino acids 329 to 521 of the whole protein sequence as described in the reference database (https://www.ncbi.nlm.nih.gov/genbank/sars-cov-2-seqs/). In this case the sequence of the RBD (SEQ ID 1) for both the Italian and Chinese Variant are identical. However, modifications are possible and well within the scope of this invention.

The skilled person will appreciate that modifications of the sequence may be possible or even desirable. While in some cases, exact structural similarity, e.g. accurate folding of the protein attached to a liposome’s surface, is necessary for the immunogenic response, in other cases, deviations are well acceptable. For example, cysteine in the peptide chain may be replaced in part or in full by serine in view of the expression system, which might e.g. be chosen to be an E.coli host organism. Disulphide bonds and, accordingly, folding of certain protein domains can sometimes be dispensed with for the purposes of a vaccine. In this case, the RBD used as an antigenic moiety may have the sequence SEQ ID 2 (Cys > Ser replacements).

One aspect of the invention relates to an immunogenic composition as described above, wherein the antigenic moiety (a) is or comprises an amino acid sequence corresponding to
(i) the HR domain of S₂ subunit of spike protein S of SARS-CoV-2 virus;
(ii) an immunogenic fragment of the said receptor binding domain RBD;
(iii) a sequence having at least 95%, preferably at least 98%, more preferably at least 99% sequence identity to the receptor binding domain RBD of Spike protein S of SARS-CoV-2 virus.

By HR domain (the postfusion core) of S₂ subunit is meant an amino acid sequence (AAs 912 to 1202) which forms part of structural spike protein S of SARS-Cov-2 virus, in particular, in sub-unit S₂ of the transmembrane spike S glycoprotein. This section has turned out to be characteristic, highly preserved and therefore serves as a target for development of vaccines. The natural sequence of the polypeptide corresponds to the one as deposited in GenBank/SRA (https://www.ncbi.nlm.nih.gov/genbank/sars-cov-2-seqs/; retrieved 06.04.2020) and is given as SEQ ID 7.

The scope of the invention, however, also extends to an immunogenic fragment thereof or a sequence having at least 90%, preferably at least 95%, more preferably at least 98% sequence identity to the said receptor binding domain RBD. For example, the cysteins may be replaced by serine, hence giving a sequence of the HR domains of S₂ subunit (AAs 912 to 1202) as listed in SEQ ID 8.

HR1 and HR2 are the post fusion core of the spike S protein and are located on the spike S₂ subunit. Similar to severe acute respiratory syndrome (SARS)-CoV, SARS-CoV-2 has the ability to utilize human angiotensin-converting receptor (ACE2) as a target to infect human cells. Spike (S) protein S2 subunit plays a central role in mediating virus fusion with the host cell, in which the heptad repeat 1 (HR1) and heptad repeat 2 (HR2) can interact to form six-helical bundle, thereby bringing viral and cellular membranes in close proximity for fusion.

Good results can be obtained, if the antigenic moiety and the liposomal formulation are administered to the patient as a mixture. A further aspect of the invention, however, relates to a composition as described above, wherein the antigenic moiety is linked to the liposomal surface to give a proteoliposome.

The authors have found that particularly good results can be achieved if particles are provided that are similar to the actual virus. When liposomes are used as a carrier for the antigenic moiety to give a proteoliposome, such liposomes mimic the virus in size and composition. The surface proteins are presented in a way inspired by the virus’ structure and are more likely to produce specific antibodies and cellular immunity against immunodominant epitopes of the virus.

The antigenic moiety may be linked to the liposomal surface:
covalently, in particular by means of an EDC (1-ethyl-3-(3-dimethylaminopropyl)carbodiimide) crosslinker;
electrostatically, in particular by means of negatively charged functional groups present on the outer surface of the liposomal bilayer.
Amongst these two, electrostatic linking can be obtained in a more straightforward production process.

For example, covalent attachment of the antigenic moiety to the liposomal surface can be achieved through a EDC (1-ethyl-3-(3-dimethylaminopropyl)carbodiimide) crosslinker. Such a crosslinker can react with carboxyl groups on the one hand and amino groups on the other hand to form amide bonds. Electrostatic interactions can be formed by using outer membrane lipids having a charge, for example phospholipids, such as DSPE (1,2-Distearoyl-sn-glycero-3-phosphoethanolamine). The negatively charged heads of such phospholipids interact with positively charged proteins.

Another aspect of the invention relates to a composition as described above, wherein the antigenic moiety is anchored in the liposomal surface, in particular by means of secondary protein structures linked to the antigenic moiety. Such secondary protein structure may be a structure which exposes apolar groups towards the liposomal bilayer, in particular a α-helix of a protein. It is an advantage of the said anchoring that the secondary protein structure can be provided directly upon expression of the antigenic moiety, in particular the RBD, that it inserts reliably into to the lipid bilayer, and that subsequent surface modification steps may be minimized.

In a preferred embodiment, the lipid bilayer of the lipids in the liposomal formulation substantially consists of cholesterol, phospholipids, in particular phosphatidylcholine and/or phosphatidylglycerol. By the term “substantially consists of” is meant that the components together add up to a total of at least 80 wt-%, preferably at least 90 wt-%, more preferably at least 95 wt% of the lipid bilayer. Examples of phosphatidylcholine include DOPC and/or HSPC. Examples of phosphatidylglycerol include DSPG. The skilled person will acknowledge, that alternatives thereof may be used with similar effects and that variations in relative amounts are possible. However, the authors have found that relative amounts of approximately 30 to 50 wt-% of cholesterol (with regard to the total amount of liposome components) combined with approximately 50 to 70 wt-% of a phosphatidylcholine and phosphatidylglycerol mixture (with regard to the total amount of liposome components; in a wt-ratio of to 2:1 to 5:1) is particularly suitable to build a basis of the liposome.

In a preferred embodiment, the lipid bilayer of the liposome substantially consists of a combination of cholesterol, L-α-phosphatidylcholine (HSPC) and 1,2-dipalmitoyl-sn-glycerol-3-phosphate (sodium salt) (DPPA). L-α-phosphatidylcholine is also known as Hydro Soy PC, hydrogenated soy or HSPC. 1,2-dipalmitoyl-sn-glycerol-3-phosphate is also known as dipalmitoylphosphatidic acid or DPPA. DPPA has been found to be able to provide suitable physiochemical properties, in particular suitable functional groups on the outer surface of the liposomal bilayer for electrostatically interacting with an antigenic moiety.

It is particularly preferred that cholesterol, phospholipid and phosphatidylglycerol are present in an amount of 25 to 40 mol-% each, preferably 30 to 35 mol-% each. It has been shown that a composition having such molar ratio provides for good liposome stability despite the sterical challenges associated with the use of DPPA.

The liposomes may be modified by PEGylation. By “PEGylation” is meant polyethylene glycol-modification. This may be performed as a post-modification or pre-modification method. Traditionally, phosphatidylcholine, cholesterol and PEG-lipid are dissolved in the same mixture to yield crude liposomes which are downsized later on by extrusion. This method is called the pre-modification method. In contrast, when applying the post-modification method, bare liposomes composed of phosphatidylcholine and cholesterol are prepared in solvent and are then extruded through suitable membrane(s) and/or are -which is preferred - formed by sonication. Only after the steps of liposome formation/minimization is a PEG-lipid added, preferably in aqueous solution. The method is described in detail in Nakamura, K.; Comparative studies of polyethylene glycol-modified liposomes prepared using different PEGmodification methods; Biochim Biophys Acta, 1818 (2012) 2801-2807. A preferred amount of PEG-lipid in the total weight of the liposomes would range between 0.5 and 5 wt-%.

It is preferred that the liposome additionally comprises at least one more adjuvant compound having a lipophilic moiety, preferably Saponin (QuilA or QS-21), Mannide mono oleate (MMO), monophosphoryl lipid A derived motive from lipopolysaccharide (MPLA LPS) or any combination thereof. While cholesterol and phospholipids account for stability of the formed liposomes and adjuvant activity, especially when injected subcutaneously or intramuscularly, Saponin, MMO and/or MPLA LPS may be added for their excellent immunostimulatory properties in vaccines for humans and animals. The adjuvant compounds having a lipophilic moiety may be added in small amounts of < 3 wt-% preferably < 2 wt-% more preferably <1 wt-% of the overall liposomal composition. It is the purpose of these adjuvants to stimulate the immune response, while being physiologically well tolerated, in contrast to traditionally used adjuvants which were often based on aluminum and suspected to be toxic.

It is particularly preferred that the adjuvant compound is Monophosphoryl Lipid A (MPLA). MPLA is the natural ligand of the Toll-like receptor 4 (TLR4). Receptor ligation induces potent T cell (Th1) and antibody responses. MPLA has been shown to be a safe and effective adjuvant, is licensed for human application in Europe and is used in vaccines as part of the Adjuvant System 04 (AS04). An MPLA-modified liposomal carrier loaded with an antigenic moiety as described above exhibits a favorable immunogenicity profile compared to the non-adjuvanted liposome. This is exemplified by increased surface expression of H2Kb (MHCII, antigen presentation) and increased production of proinflammatory cytokines IL-6 and IL-12 by in vitro stimulated dendritic cells (DCs). Additionally, both the liposomal base vesicle and the MPLA-adjuvanted liposome induced the expression of co-stimulatory molecules CD80, CD83 and CD40 on treated DCs in vitro.

In an embodiment of the invention, where the antigenic moiety is covalently linked to the liposomal surface to give a proteoliposome, the covalent bond between the liposome and the antigenic moiety may been derived from lipid bilayer components having reactive head moieties, preferably DSPE or stearic acid. In this case, DSPE and/or stearic acid are incorporated into the liposomal bilayer in an amount of approximately 1 to 5 wt-% of the total liposome components. Such reactive heads, comprising carboxyl groups or amino groups respectively, may react for example via an EDC crosslinker with a complementary amino group / carboxyl group of the antigenic peptide, to eventually, form an amide bond.

In an embodiment of the invention, where there is an electrostatic interaction between the liposome and the antigenic moiety, the interaction may be established due to lipid bilayer components having charged head moieties. Examples of lipid bilayer components having charged head moieties include DSPG (1,2-Distearoyl-sn-glycero-3-phosphoglycerol) and DOTAP (1,2-dioleoyl-3-trimethylammonium-propane). The choice of charged head moiety depends on the isoelectric point of protein and the pH with which the composition is applied. The RBD of Spike S protein has a theoretical pI of 8.67 and thus at pH 6.8 to 7.0 carries a positive charge. There might be a need for a negatively charged lipid for the specific conditions. Other domains of interest, such as the core protein domain of Spike S2 might have a different pI (e.g. 5.4) and will thus carry a negative charge at a pH between 6.8 and 7.0. In such a case, a positively charged lipid head will be preferred. In the case of Spike S2 core protein, for example, the theoretical pI is 5.4 and the protein is negative at pH 6.8 to 7.0, so there is a need of a positively charged lipid such as DOTAP.

In a preferred embodiment of the invention, the base vesicles display a surface charge of -50 to -60 mV, preferably -53 to -57 mV (Zeta-Potential). For example, where the lipid bilayer of the liposome in formulation consists of a combination of cholesterol, HSPC and DPPA, in a (preferable) molar ratio of substantially 1:1:1, the resulting Zeta-Potential typically is -55 mV. The Zeta-potential can be analysed by using DLS. Such Zeta potential is particularly suitable for electrostatically interacting with an immunogenic fragment of the RBD domain as described above. The protein antigens (RBD, but also S1, ECD) are adsorbed to the liposomal surface as indicated by an increase in the hydrodynamic diameter of liposomes combined with the protein antigen.

It is preferred that the liposomes of the formulation as described above have a mean diameter between 10 and 120 nm, preferably between 20 and 100 nm, measured by dynamic light scattering DLS. More preferred are liposomes having an average diameter (Z. Ave.) of 60 to 100 nm, preferably 70 to 90 nm, measured by dynamic light scattering DLS. Such sizes best mimic the size of the template corona virus. The morphology and size of the liposomes of the formulation can be varied according to the teachings of WO2020/254633A1, the contents of which are included herein by reference. In a preferred embodiment, the liposomal dispersion has a polydispersitiy index (PDI) of ≤ 0.15, preferably ≤ 0.10, more preferably ≤ 0.09, measured by dynamic light scattering (DLS).

For the purposes of this invention, the “base vesicle” is understood as the liposome formed by cholesterol and phospholipids only, preferably formed by cholesterol, phosphatidylcholine and dipalmitoylphosphatidic acid, i.e. before adding further modifications or insertions such as MPLA insertion, PEGylations, etc. The surface charge, size, polydispersity and stability of the liposomal dispersion were experimentally determined for the base vesicles. However, it can be expected that the values do not substantially change upon surface modification.

For the purposes of this invention, “measured by dynamic light scattering” (DLS) means that DLS was performed on samples diluted in PBS or MQ H₂O to reach an attenuation factor in the instrument of 6. DLS was measured on a Malvern Zetasizer Nano device at 25° C. and 0° scattering angle. Instrument control and data analysis were performed with the Zetasizer software (version 7.11) from Malvern. Particle size (hydrodynamic diameter) was determined using the Stokes-Einstein equation:

$$d \left(H\right) = \frac{k T}{3 π η D}$$

where k is Bolzmann’s constant; T is absolute temperature; η is dispersant viscosity and D is diffusion coefficient. Viscosity was determined with the Zetasizer software and was 0.8872 cP. Dispersant refractive index was 1.330. D was obtained by fitting the autocorrelation function with a suitable algorithm. Cumulants analysis is a simple method of analysing the autocorrelation function generated by a DLS experiment and produces the mean particle size (Z.Ave.) and polydispersity index (PDI). The calculation is defined in ISO 13321 (1996) and ISO 22412 (2008). The first order result from a DLS experiment is an intensity distribution of particle sizes. The intensity distribution is naturally weighted according to the scattering intensity. The size distribution is displayed as a plot of the relative intensity of light scattered by particles (on the Y axis) versus various size classes (on the X axis) which are logarithmically spaced. Clear disposable zeta cells with a pathlength of 10 mm were used for the measurements.

The size of the liposome has important effects on the immune response provoked. It has been generally assumed in the literature that the size of liposomes determines, whether the development of Th2 responses or Th1 responses are enhanced. It has been furthermore suggested that a certain (large) size is required for particles to be trapped in tissues, creating depot effects in the injection site to recruit and be phagocytosed by APCs, which will then mature and travel to draining lymph nodes for inducing immune responses. It has now been found by the authors that liposomes sized 60 to 100 nm, preferably 75 to 85 nm are particularly useful for delivering a specific antigenic moiety as described above, while having proven effects as a potent immunologic adjuvant.

A polydispersity index ≤ 0.15 is superior over the polydispersity indices of liposomal formulations known in the art. Liposomal formulations known in the art, available by extrusion, homogenization, and sonication procedures, typically show polydispersity indices of 0.2 to 0.4 (Gim Ming Ong et al., Evaluation of Extrusion Technique for Nanosizing Liposomes, Pharmaceutics 2016 (8) 36, p. 5). Essentially monodisperse liposomal formulations are beneficial for reproducibility purposes, industrial scale production and compliant with marketing authorization requirements. They are apt for sterile filtration and display high stability over time.

A further aspect of the invention relates to a method of producing an immunogenic composition as described above, comprising the step of combining
(a) the antigenic moiety and
(b) the liposomal formulation.
As described above, the components may be combined by admixing or mixing. However, the components may also be attached to each other, either covalently or electrostatically or by means of lipophilic interaction. The preferred liposomal compositions and exemplary linking mechanisms have been described above.

It is preferred that, prior to combining (a) the antigenic moiety and (b) the liposomal formulation, there are steps of
providing lipids in a mixture of organic solvent and an aqueous liquid,
sonicating to enable liposome formation.
Producing the liposomes by means of sonication, is a simple and reliable way of obtaining liposomes which are less polydisperse, more stable and less prone to degradation than liposomes obtainable by conventional techniques, in particular liposomes obtainable by extrusion techniques. An excellent method for providing high quality liposomes has been described in WO2020/254633A1, the contents of which are included herein by reference. It is particularly preferred that the method does not contain any extrusion step or any thin film hydration step.

It is preferred that in the method as described above, the sonication step is followed by a step of inserting MPLA into the liposomal lipid bilayer, preferably by means of heat insertion. The skilled person will be aware that the insertion may or may not follow immediately after the liposome formation. Intermediate steps, such as purification, in particular filtration, are possible and encompassed.

It is preferred that in the method as described above, prior to combining (a) the antigenic moiety and (b) the liposomal formulation, there are performed the steps of:
providing a gene sequence encoding for
   (i) the receptor binding domain RBD of Spike protein S of SARS-CoV-2 virus;
   (ii) an immunogenic fragment of the said receptor binding domain RBD;
   (iii) a sequence having at least 90%, preferably at least 95%, more preferably at least 98% sequence identity to the receptor binding domain RBD of Spike S protein of SARS-CoV-2 virus;
expressing the genetic sequence in an expression host organism, preferably a human cell expression system, to give (a) the antigenic moiety.

A gene sequence encoding for the receptor binding domain RBD of Spike protein S of SARS-CoV-2 virus, in particular amino acids 329 to 521, may be provided as SEQ ID 3 (Gene Sequence encoding for SEQ ID 1).

The skilled person will appreciate that variations are well possible, even likely and desirable. The sequence may provide for modifications such as Cys > Ser replacements. In this case, the sequence might be provided as SEQ ID 4 (Gene Sequence Encoding for SEQ ID 2).

The genetic code may be chosen to improve efficiency of expression or may be to optimized in view of the respective expression host organism. The genetic code may be provided on plasmid.

An expression host organism may be E.coli. Expression in E.coli is well established, efficient and sufficiently ensures sequence mediated immune responses. However, other preferred expression host organisms include baculovirus, yeast, mammal cells or insect cells which might better provide for glycosylation, cysteine expression and, as a result, protein folding for a structural and sequence mediated immune response. Experiments have shown that expression is most efficient in a human cell expression system, such as HEK293, since it ensures proper protein folding and correct post-translational modifications.

After expression the amino acid sequence may be purified by chromatographic methods, e.g. 8x His-Tag affinity chromatography, and the His-Tags may then be removed at previously introduced TEV-cleavage sites.

The invention further relates to an immunogenic composition as described above for use as a medicament. In particular, the immunogenic composition is for use as a vaccine in the prevention of COVID-19. In the latter case, one vaccine shot may preferably comprise an amount of 10 to 100 µg of protein sequence.

The invention will be better understood by means of the following examples. They are not meant, however, to limit the scope of the invention.

The figures are briefly described hereinafter:

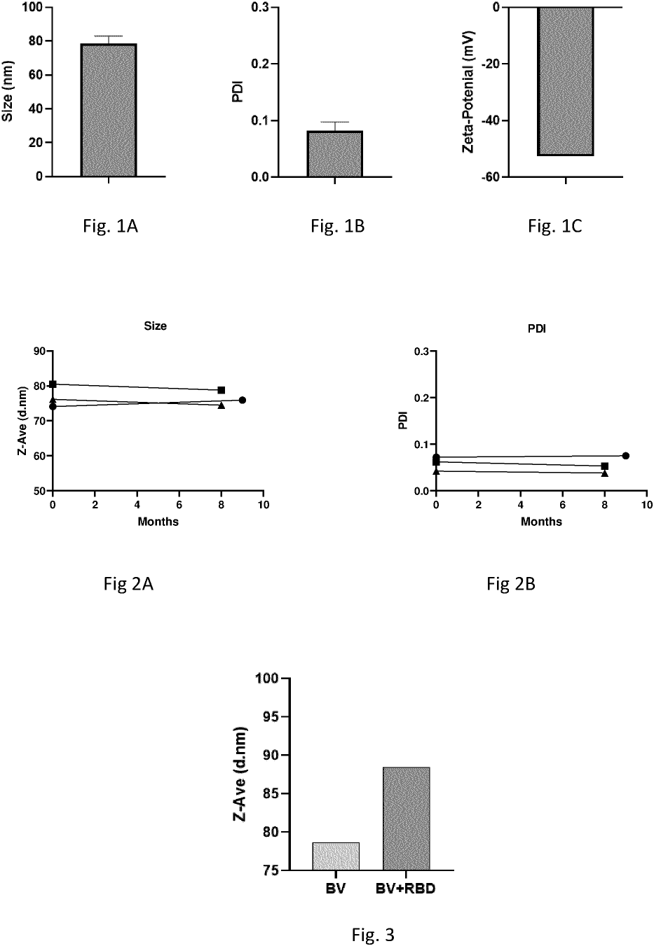

FIGS. 1A-C: Representation of liposome base vesicle size / PDI / Zeta-Potential measurements;

FIGS. 2A-B: Stability of liposome base vesicle size and PDI in three batches over time;

FIG. 3: Representation of mean particle size (Z.Ave.) of an exemplary base vesicle before and after load with RBD protein;

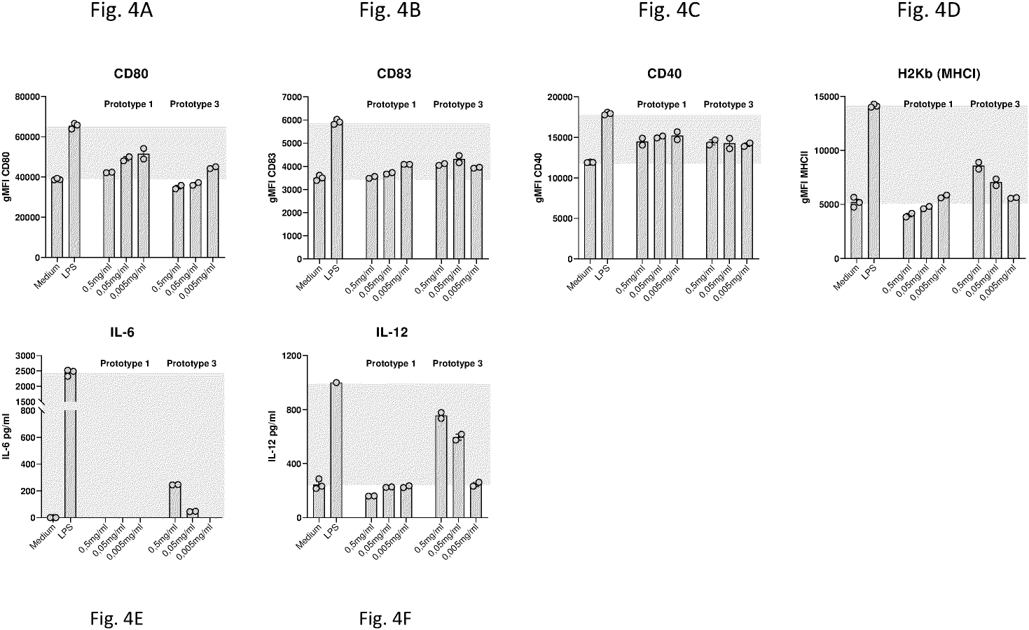

FIGS. 4A-F: Results of in vitro studies regarding the immunogenicity profile of exemplary vesicles;

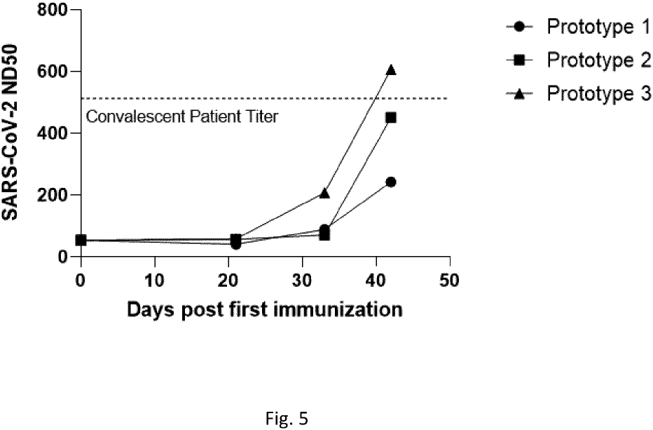

FIG. 5: Results of SARS-CoV-2 neutralizing assay in mice after immunization with the vaccine.

### EXAMPLE 1: EXPRESSION OF AN ANTIGENIC MOIETY

A clone comprising SEQ ID 4 was provided in a plasmid, added into Bacteria BL21 E. Coli strain, and expressed to obtain a sufficient peptide amount. The bacteria were collected by centrifugation and subject to lysis. The expressed protein was purified by ion exchange or affinity chromatography (His Tag 8x), the His-Tags were removed by means of TEV protease, followed by an additional purification step based on size exclusion chromatography. Alternatively, a clone comprising SEQ ID 4 can be provided in a plasmid, added into a human cell expression system (HEK293) and expressed to obtain a sufficient peptide amount.

### EXAMPLE 2: PREPARATION OF A LIPOSOMAL FORMULATION FOR ADMIXING WITH THE ANTIGENIC MOIETY

40 mol% Cholesterol and 60 mol% HSPC were mixed in prewarmed ethanol prior to being added to aqueous solution (0.9% NaCL 20 mM Hepes pH, 6.8). After dissolving the solvent and aqueous part, the mixture was sonicated for up to 4 hours at a temperature between 60 and 70° C. After liposomes reached an optimal size distribution (polydispersity index PDI of max. 0.12) sonication was stopped and the solution cooled down to 22° C. and purified by filtration.

### EXAMPLE 3: ADMIXING OF ANTIGENIC MOIETY AND LIPOSOMAL FORMULATION

Purified protein according to Example 1 and the liposomal formulation according to Example 2 were added to give a final protein concentration of 20 µg/ml and final total lipid concentration of 5 mg/ml.

### EXAMPLE 4: PREPARATION OF A LIPOSOMAL FORMULATION FOR ADMIXING WITH THE ANTIGENIC MOIETY

30 mol% cholesterol, 66 to 69 mol% HSPC and 1 mol% MMO were mixed and processed to give a liposomal formulation, following the procedure as described in Example 2. Subsequently, 2 mol% DSPE-PEG2000 were added and incorporated by postinsertion method (described in WO2020/254633A1). The mixture was heated to 65° for 1 h. Purified protein was obtained as described in Example 1. Protein and liposomal formulation were combined by admixing, to give a final protein concentration of 30 µg/ml and a final total lipid concentration of 10 mg/ml.

### EXAMPLE 5A: PREPARATION OF A LIPOSOMAL FORMULATION FOR ELECTROSTATICALLY INTERACT WITH THE ANTIGENIC MOIETY

30 mol% cholesterol, 49% mol% HSPC, 20 mol% DSPG, 0.5 mol% saponin Qs-21 and 0.5 mol% MMO were mixed and processed to give a liposomal formulation, following the procedure as described in Example 2. Purified protein was obtained as described in Example 1. Protein and liposomal formulation were combined to give a final protein concentration of 15 µg/ml and a final total lipid concentration of 10 mg/ml. The pH of the composition was adjusted to physiologic conditions (pH 6.8-7.0) and due to the negative charge of the DSPG and the positive total charge of RBD protein, electrostatic interactions can be established.

### EXAMPLE 5B: PREPARATION OF A LIPOSOMAL FORMULATION FOR ELECTROSTATICALLY INTERACTING WITH THE ANTIGENIC MOIETY

33% HSPC, 33% DPPA and 33% Cholesterol were mixed in pre-warmed ethanol prior to being added to aqueous solution (0.9% NaCL 10 mM Hepes pH, 6.8). After dissolving the solvent and aqueous part, the mixture was sonicated for up to 4 hours at a temperature between 60 and 70° C. After liposomes reached an optimal size distribution, sonication was stopped, and the solution cooled down to 22° C. and purified by filtration.

The liposomes were measured to display an average size of 80 nm (FIG. 1A), a PDI of 0.9 (FIG. 1B) and a Zeta-Potential of -55 mV (FIG. 1C).

Stability of the base vesicles thus obtained was monitored over time (up to 9 months) for three batches, each obtained as described above. The base vesicle formulation was found to be extraordinarily stable, with hardly any deviation in either size or PDI (FIGS. 2A and 2B, square/triangle/circle data each represent a production batch).

### EXAMPLE 5C: HEAT-INSERTION OF MPLA

After the formation of the liposomal base vesicles as described above (Example 5b), the adjuvant molecule MPLA was inserted into the liposomal lipid bilayer via heat-insertion. MPLA was dissolved in HEPES (10 mM, 0.9% NaCl, pH 6.8) at a concentration corresponding to 0.3 mol-% of the total lipid concentration in the base vesicle. MPLA and base vesicle solution obtained in Example 5b were mixed and incubated for 30 min at 65° C. Sterile filtation of the solution.

### EXAMPLE 5D: COMBINATION OF LIPOSOMAL DISPERSION AND RBD

Purified protein was obtained as described in Example 1. Protein and liposomal formulation obtained in example 5c were combined to give a final protein concentration of 10 µg/100 µl and a final total lipid concentration of 20 mg/ml. The pH of the composition was adjusted to physiologic conditions (pH 6.8-7.0) and due to the negative charge of the DPPA and the positive total charge of RBD protein, electrostatic interactions were established. The protein antigens were adsorbed to the liposomal surface as indicated by an increase in the hydrodynamic diameter of liposomes combined with the protein antigen. This is visible in FIG. 3 (BV: base vesicle).

### EXAMPLE 6: PREPARATION OF A LIPOSOMAL FORMULATION FOR COVALENT BONDING TO AN ANTIGENIC MOIETY

40 mol% cholesterol, 39% mol% HSPC, 20 mol% stearic acid 1 mol% saponin QS-21 and 0.5 mol% MMO were mixed and processed to give a liposomal formulation, following the procedure as described in Example 2. Purified protein was obtained as described in Example 1. Protein and liposomal formulation were combined, 6 mM EDClinker was added and the reaction mixture was incubated overnight.

### EXAMPLE 7: IN VITRO STUDIES OF IMMUNOGENICITY PROFILE

The immunogenicity profile of the liposomal carrier was assessed using an in vitro DC-maturation assay (DC: dendritic cell). “Prototype 1” is a liposomal formulation obtained as described in example 5b, “Prototype 3” is a liposomal formulation obtained as described in example 5c, hence an MPLA-adjuvanted liposomal carrier. The latter (Prototype 3) exhibits a favorable immunogenicity profile compared to the non-adjuvanted liposome, as can be seen from FIGS. 4A to 4F, where “Medium” is the unstimulated control group and LPS (Lipopolysaccharide) is the benchmark. Prototype 1 exhibits some DC activating activity, as indicated by elevated surface expression of CD80, CD83 and CD40 compared to the unstimulated (medium) condition. However, only stimulation of DC with MPLA modified liposomal carriers resulted in a pronounced upregulation of MHCI (antigen cross-presentation) and secretion of IL-6 and IL-12. These data indicate that incorporation of MPLA to the liposomal carrier is beneficial in order to induce a pronounced adaptive immune response resulting in the generation of SARS-CoV-2 neutralizing antibodies.

### EXAMPLE 8: IMMUNIZATION WITH THE VACCINE INDUCES SARS-COV-2 NEUTRALIZING ANTIBODY TITERS

The capacity of the vaccine prototypes to induce SARS-CoV-2 neutralizing antibodies was tested by immunizing BALB/c mice, as follows: Three subsequent immunization treatments were performed on day 0, day 21 and day 33 in each case, applying each time a dosage of 100 µl/animal, 10 or 30 µg RBD/dose (intra-muscular administration).

The results are displayed in FIG. 5. Three prototypes were tested in the immunization studies. Those were
(1) Prototype 1: base vesicle loaded with 10 µg/100 µl RBD,
(2) Prototype 2: base vesicle loaded with 30 ug/100 ul RBD, and
(3) Prototype 3: MPLA-modified base vesicle loaded with 10 µg/100 µl RBD.

Immune sera of immunized animals were collected at days 21, 27 and 42 post primary immunization. SARS-CoV-2 neutralizing antibody titers were determined in vitro, utilizing a SARS-CoV-2 neutralization assay. As shown in FIG. 5, immunization with all three prototypes induced detectable SARS-CoV-2 neutralizing antibody titers. Immunization with Prototype 3 was most potent and resulted in SARS-CoV-2 neutralizing titers similar to those observed in COVID-19 convalescent patients.

1. An immunogenic composition comprising:
(a) an antigenic moiety; and
(b) a liposomal formulation as an adjuvant.

2. The immunogenic composition according to claim 1, wherein the antigenic moiety (a) is or comprises an amino acid sequence corresponding to
(i) the receptor binding domain RBD of Spike protein S of SARS-CoV-2 virus;
(ii) an immunogenic fragment of said receptor binding domain RBD; or
(iii) a sequence having at least 90%, sequence identity to the said receptor binding domain RBD.

3. The composition according to claim 1, wherein the antigenic moiety is or comprises an amino acid sequence selected from the group consisting of:
(i) SEQ ID NO 1;
(ii) an immunogenic fragment of SEQ ID NO 1; and
(iii) a sequence having at least 90%, at sequence identity to SEQ ID No 1.

4. The immunogenic composition according to claim 1, wherein the antigenic moiety (a) is or comprises an amino acid sequence corresponding to at least one of:
(i) the HR domain of S₂ subunit of spike protein S of SARS-CoV-2 virus;
(ii) an immunogenic fragment of said receptor binding domain RBD; and
(iii) a sequence having at least 95% sequence identity to said receptor binding domain RBD;.

5. The composition according to claim 1, wherein the antigenic moiety is linked to the liposomal surface of said liposomal formulation to give a proteoliposome.

6. The composition according to claim 1, wherein the antigenic moiety is linked to the liposomal surface of said liposomal formulation covalently or electrostatically.

7. The composition according to claim 1, wherein a lipid bilayer of the liposomal formulation substantially consists of a combination of cholesterol and at least one of phosphatidylcholine and phosphatidylglycerol.

8. The composition according to claim 7, wherein the lipid bilayer of the liposomal formulation substantially consists of a combination of cholesterol, L-α-phosphatidylcholine (HSPC) and 1,2-dipalmitoyl-sn-glycerol-3-phosphate (salt; DPPA).

9. (canceled)

10. The composition according to claim 7, wherein the composition additionally comprises Saponin (QuilA or QS-21), Mannide mono oleate (MMO), monophosphoryl lipid A derived motive from lipopolysaccharide (MPLA LPS) or any combination thereof.

11-13. (canceled)

14. The composition according to claim 6, wherein the liposomes of said liposomal formulation have a surface charge of -50 to -60 mV (Zeta-Potential).

15. The composition according to claim 1, wherein the liposomes of the liposomal formulation have an average diameter (Z. Ave.) of 60 to 100 nm, measured by dynamic light scattering DLS.

16. (canceled)

17. A method of producing an immunogenic composition according to claim 1, the method comprising the step of combining
(a) the antigenic moiety and
(b) the liposomal formulation.

18. The method according to claim 17, further comprising, prior to combining (a) the antigenic moiety and (b) the liposomal formulation, the steps of:
providing lipids in a mixture of organic solvent and an aqueous liquid; and
sonicating to enable liposome formation.

19. The method according to claim 18, wherein the sonication step is followed by:
inserting MPLA into the liposomal lipid bilayer.

20. The method according to claim 17, further comprising, prior to combining (a) the antigenic moiety and (b) the liposomal formulation, the steps of:
providing a gene sequence encoding for
(i) the receptor binding domain RBD of Spike protein S of SARS-CoV-2 virus;
(ii) an immunogenic fragment of said receptor binding domain RBD; and/or
(iii) a sequence having at least 90%, sequence identity to the receptor binding domain RBD of Spike protein S of SARS-CoV-2 virus; and
expressing the genetic sequence in an expression host organism, to give (a) the antigenic moiety.

21. An immunogenic composition according to claim 1 for use as a medicament.

22. An immunogenic composition according to claim 1 for use as a vaccine in the prevention of COVID-19.

23. The immunogenic composition according to claim 1, wherein said antigenic moiety is or comprises an amino acid sequence corresponding to a surface protein domain of SARS-CoV-2 virus.

24. The immunogenic composition according to claim 6, wherein covalent linkage takes place by means of EDC (1-ethyl-3-(3-dimethylaminopropyl)carbodiimide) crosslinker, or wherein electrostatic linkage takes place by means of negatively charged functional groups present on the outer surface of the liposomal bilayer.
